(19) United States
(12) Patent Application Publication
(10) Pub. No.: US 2005/0074420 A1
(43) Pub. Date: Apr. 7, 2005

(54) Composition in the form of a W/O emulsion

(75) Inventors: Audrey Bourdel, Rueil Malmaison (FR); Emmanuelle Labrousse, Igny (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/921,991

(22) Filed: Aug. 20, 2004

Related U.S. Application Data
(60) Provisional application No. 60/507,087, filed on Oct. 1, 2003.

(30) Foreign Application Priority Data
Sep. 12, 2003 (FR) 0310750

(51) Int. Cl. A61K 7/06; A61K 7/11

(52) U.S. Cl. 424/70.12

(57) ABSTRACT

A composition in the form of a water-in-oil emulsion having an aqueous phase dispersed in an oily phase, and containing at least one silicone oil, at least one silicone emulsifier, at least one grafted silicone polymer comprising a polysiloxane portion and a non-silicone organic chain portion, one of the two portions constituting the main chain of the polymer and the other being grafted onto the main chain, at least 15% by weight of at least one C₂-C₃ primary alcohol relative to the total weight of the composition, and at least 5% by weight of one or more polyols relative to the total weight of the composition.

### REFERENCE TO PRIOR APPLICATIONS

This application claims priority to U.S. provisional application 60/507,087 filed Oct. 1, 2003, and to French patent application 0310750 filed Sep. 12, 2003, both incorporated herein by reference.

### FIELD OF THE INVENTION

The present invention relates to a composition in the form of a water-in-oil (W/O) emulsion, comprising a primary alcohol, a volatile silicone oil, polyol(s), a silicone surfactant and a grafted silicone polymer. This composition may be used in particular in cosmetics and/or dermatology, more particularly in cosmetics to give a body composition that has a coating effect.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The skin is an organ that is essential for the body, and it is common practice to use body products intended to constitute a protective film and to protect human body skin against dehydration and dryness. These products are generally in the form of oil-in-water (O/W) emulsions, since, due to the fact that they comprise an outer aqueous phase, these emulsions have the advantage of feeling fresh when applied to the skin. However, these emulsions may have an insufficient protective effect against dehydration.

Thus, it is increasingly sought to use water-in-oil (W/O) emulsions since these emulsions, which comprise an oily continuous outer phase, allow a lipid film to be formed on the surface of the skin, and thus to prevent transepidermal water loss and to protect the skin against external attacks. Thus, these emulsions are particularly suitable for protecting and nourishing human body skin. However, compositions in the form of W/O emulsions have the drawback of giving a quite greasy feel when applied to the skin, due, precisely, to the fact that the oily phase is the outer phase, and thus lacks freshness. Hence, these creams are generally used for dry skin, since they are too greasy to be used on greasy skin. Furthermore, W/O emulsions are generally too rich in oils to be used during summer or in countries with a hot climate.

To overcome these drawbacks, it has been envisaged to prepare W/O emulsions containing alcohol. However, the alcohol content cannot be too large for reasons of tolerance, since the presence of large amounts of alcohol (greater than 40%) may lead to skin irritation problems, especially in the case of individuals with sensitive skin. Furthermore, the presence of a large amount of alcohol may cause additional problems, especially in terms of stability of the composition.

There is thus still a need for a topical composition for the body that provides good protection on the skin while at the same time feeling fresh when applied.

### DETAILED DESCRIPTION OF THE PREFERRED EMBODIMENTS

The inventors have discovered, entirely fortuitously, that the use of a particular silicone grafted polymer and also particular proportions of alcohol and polyol(s) makes it possible to overcome the drawbacks of the prior art and to obtain compositions in the form of water-in-oil emulsions that have the desired properties.

One subject of the present invention is thus a composition, preferably suitable for topical application, in the form of a water-in-oil emulsion comprising an aqueous phase dispersed in an oily phase, and:
at least one silicone oil,
at least one silicone emulsifier,
at least one grafted silicone polymer comprising a polysiloxane portion and a non-silicone organic chain portion, one of the two portions constituting the main chain of the polymer and the other being grafted onto the main chain,
at least 15% by weight of at least one C₂-C₃ primary alcohol relative to the total weight of the composition, and
at least 5% by weight of one or more polyols relative to the total weight of the composition.

Since the composition according to the invention is intended for topical application, it preferably constitutes a physiologically acceptable medium. The expression “physiologically acceptable medium” means a medium that is compatible with the skin, the lips, the nails, the scalp and/or the hair. This composition may especially constitute a cosmetic composition.

The composition according to the invention has good skin-protecting properties and especially coating properties, while at the same time feeling fresh when applied. The term “coating properties” means herein the fact that the composition of the invention gives the sensation, after it has been applied to the skin, of forming a coat on the skin and of thus acting as a second skin. For example, after application to the legs, the user has the impression of wearing a stocking. The composition according to the invention is also very comfortable. In addition, the film formed on the surface of the skin after applying this composition allows clothes to be put on more easily.

The composition according to the invention is preferably generally translucent. The transparency or translucency of a composition may be measured using a turbidimeter. The model 2100P® portable turbidimeter from the company Hach may be used, for example, to measure the transparency ranges of a composition. The composition is generally considered as translucent for a turbidity value ranging from 250 to 1000 NTU, including 300, 400, 500, 600, 700, 800, and 900 NTU.

Primary Alcohol

The term “C₂-C₃ primary alcohol” means a linear or branched aliphatic lower monoalcohol containing 2 or 3 carbon atoms. According to one preferred embodiment of the invention, the primary alcohol used is ethanol. The alcohol is generally present in an amount of at least 15% by weight relative to the total weight of the composition. This amount may range, for example, from 15% to 40% by weight and better still from 18% to 30% by weight relative to the total weight of the composition, including 20, 22, 24, 26, and 28%.

Silicone Emulsifier

Examples of silicone emulsifiers that may be mentioned include (1) dimethicone copolyols such as those sold under the names DC 5329, DC 7439-146 and DC 2-5695 by the company Dow Corning, the mixture of cyclomethicone and of dimethicone copolyol (CTFA name: Cyclopentasiloxane/PEG/PPG-18/18 Dimethicone), sold under the names DC 3225 C, DC 5225 C and DC 5185 C by the company Dow Corning, or the product sold under the name Abil EM 97 by the company Goldschmidt, and (2) alkyldimethicone copolyols, such as the laurylmethicone copolyol sold under the name Dow Corning 5200 Formulation Aid by the company Dow Corning, and the cetyldimethicone copolyol sold under the name Abil EM 90 by the company Goldschmidt, or the polyglyceryl-4 isostearate/cetyldimethicone copolyol/hexyl laurate mixture sold under the name Abil WE 09 by the company Goldschmidt, and mixtures thereof.

One or more co-emulsifiers may be added to the silicone emulsifier. Advantageously, the co-emulsifier may be chosen from the group comprising polyol alkyl esters. Polyol alkyl esters that may especially be mentioned include glycerol and/or sorbitan esters and for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

The amount of silicone emulsifier (as active material) may range, for example, from 0.1% to 5% by weight, preferably from 0.5% to 3% by weight and better still from 1% to 1.5% by weight relative to the total weight of the composition. The amount of co-emulsifier may also range, for example, from 0.1% to 5% by weight, preferably from 0.5% to 3% by weight and better still from 1% to 1.5% by weight relative to the total weight of the composition.

Grafted Silicone Polymer

According to the present invention, the expression “grafted silicone polymer” means a polymer comprising a main silicone chain (or polysiloxane (≡Si—O—)ₙ) onto which is grafted, inside the chain as well as, optionally, on at least one of its ends, at least one non-silicone organic monomer (i.e. containing no silicone).

The polymers containing a polysiloxane skeleton grafted with non-silicone organic monomers according to the invention may be existing commercial products or may be obtained according to any means known to those skilled in the art, in particular by reaction between (i) a starting silicone which is correctly functionalized on one or more of its silicon atoms, and (ii) a non-silicone organic compound which is itself correctly functionalized with a function which is capable of reacting with the functional group(s) borne by the silicone, forming a covalent bond; a classic example of such a reaction is the hydrosilylation reaction between ≡Si—H groups and vinyl groups CH₂═CH—, or alternatively the reaction between thio functional groups —SH and these same vinyl groups.

Examples of polymers with a polysiloxane skeleton grafted by non-silicone organic monomers which are suitable for carrying out the present invention, as well as their specific mode of preparation, include those described in particular in patent applications EP-A-0 582 152, WO-A-93/23009 and WO-A-95/03776, the teachings of which are included in their entirety in the present description as non-limiting references.

According to one particularly preferred embodiment of the present invention, the grafted silicone polymer which is obtained by means of the radical copolymerization between, on the one hand, at least one non-silicone anionic organic monomer having ethylenic unsaturation and/or a non-silicone hydrophobic organic monomer having ethylenic unsaturation, and, on the other hand, a silicone having in its chain at least one functional group capable of reacting with the ethylenic unsaturations of the non-silicone monomers, forming a covalent bond, in particular thio functional groups.

According to the present invention, the anionic monomers containing ethylenic unsaturation are preferably chosen, alone or as a mixture, from linear or branched, unsaturated carboxylic acids, optionally partially or totally neutralized in the form of a salt, it being possible for this (these) unsaturated carboxylic acid(s) to be, more particularly, acrylic acid, methacrylic acid, maleic acid, maleic anhydride, itaconic acid, fumaric acid and crotonic acid. The suitable salts are, in particular, alkali metal salts or alkaline-earth metal salts and ammonium salts. It will likewise be noted that, in the final grafted silicone polymer, the organic group of anionic nature which comprises the result of the radical polymerization of at least one anionic monomer of unsaturated carboxylic acid type can, after reaction, be post-neutralized with a base (sodium hydroxide, aqueous ammonia, etc.) in order to bring it into the form of a salt.

According to the present invention, the hydrophobic monomers containing ethylenic unsaturation are preferably chosen, alone or as a mixture, from acrylic acid esters of alkanols and/or methacrylic acid esters of alkanols. The alkanols are preferably those of C₁-C₁₈ and more particularly of C₁-C₁₂. The preferred monomers are chosen from the group consisting of isooctyl (meth)acrylate, isonyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof.

According to one preferred embodiment of the invention, the grafted silicone polymer is chosen from polydimethylsiloxanes (PDMSs) onto which are grafted, via a linker of thiopropylene type, mixed polymer units of the poly(meth)acrylic acid type and/or of the polyalkyl (meth)acrylate type. These polymers are referred to under the CTFA name “Polysilicone-8”.

It may thus be a propylthio(polymethyl acrylate), propylthio(polymethyl methacrylate) and propylthio(polymethacrylic acid) grafted polydimethylsiloxane. As a variant, it may be a propylthio(polyisobutyl methacrylate) and propylthio(polymethacrylic acid) grafted polydimethylsiloxane.

Such grafted silicone polymers are sold especially by the company 3M under the trade names VS 80, VS 70 or LO21.

Preferably, the number-average molecular mass of the silicone polymers containing a polysiloxane skeleton grafted with non-silicone organic monomers of the invention ranges from 10 000 to 1 000 000 approximately and even more preferably from 10 000 to 100 000 approximately.

The amount of grafted silicone polymer (as active material) may range, for example, from 0.1% to 5% by weight and preferably from 0.1% to 3% by weight relative to the total weight of the composition.

Polyols

The composition according to the invention preferably contains at least 5% by weight of one or more polyols relative to the total weight of the composition. The term “polyol” should be understood as meaning any organic molecule comprising at least two free hydroxyl groups. Examples of polyols that may be mentioned include glycerol, glycols, for instance butylene glycol, propylene glycol, isoprene glycol, dipropylene glycol, hexylene glycol, pentylene glycol and polyethylene glycols, for instance PEG-8, sorbitol and sugars, for instance glucose, and mixtures thereof.

The amount of polyol(s) may range, for example, from 5% to 30% by weight and better still from 7% to 25% by weight relative to the total weight of the composition, including 10, 12, 14, 16, 18, 20, 22 and 24%.

Oily Phase

The fatty phase or oily phase of the emulsion according to the invention comprises at least one silicone oil, preferably at least one volatile silicone oil.

The silicone oil may originate with the oil present in the mixture with the silicone surfactant used and/or of oils added to the composition. It may be volatile or non-volatile.

The volatile silicone oil may be chosen, for example, from cyclic or substantially linear polydimethylsiloxanes, and mixtures thereof. The cyclic polydimethylsiloxanes, or cyclomethicones, contain from about 3 to 9 silicon atoms and preferably from 4 to 6 silicon atoms and may be, for example, cyclohexadimethylsiloxane and cyclopentadimethylsiloxane. The volatile substantially linear polydimethylsiloxanes preferably contain from about 3 to 9 silicon atoms. The volatile, substantially linear polydimethylsiloxanes generally have a viscosity at 25° C. of less than or equal to 5 cSt, while the cyclomethicones generally have a viscosity at 25° C. of less than or equal to 10 cSt.

Examples of non-volatile silicone oils that may be mentioned include phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethyl siloxysilicates, and mixtures thereof.

The amount of (volatile and non-volatile) silicone oil(s) may range, for example, from 5% to 55% by weight, preferably from 10% to 50% by weight and better still from 10% to 40% by weight, including 15, 20, 25, 30 and 35%, relative to the total weight of the composition. The volatile silicone oils may constitute all or some of this amount.

According to one preferred embodiment of the invention, the total amount of polyols and of silicone oils ranges from 25% to 60% by weight and better still from 30% to 50% by weight, including 35, 40, and 45%, relative to the total weight of the composition.

Besides the silicone oils, the composition according to the invention may contain non-silicone oils. However, these oils should be in a proportion such that they do not adversely affect the advantageous properties of the composition according to the invention, especially the pleasant feel and the lightness. They are thus generally in small amount, preferably in an amount of less than or equal to 5% relative to the total weight of the composition and better still in an amount of less than or equal to 3% relative to the total weight of the composition. As non-silicone oils that may be used in the composition of the invention, examples that may be mentioned include:
hydrocarbon-based oils of animal origin, such as perhydrosqualene;
hydrocarbon-based oils of plant origin, such as sunflower oil, jojoba oil or shea butter oil;
synthetic esters and ethers, especially of fatty acids;
linear or branched hydrocarbons of mineral or synthetic origin;
fatty alcohols containing from 8 to 26 carbon atoms, for instance cetyl alcohol, stearyl alcohol and the mixture thereof (cetylstearyl alcohol);
fluoro oils;
and mixtures thereof.

The term “hydrocarbon-based oil” in the list of oils mentioned above means any oil predominantly comprising carbon and hydrogen atoms, and possibly ester, ether, fluoro, carboxylic acid and/or alcohol groups.

According to one preferred embodiment of the invention, the oily phase comprises, as oils, only silicone oils.

The other fatty substances that may be present in the oily phase are, for example, gums such as silicone gums.

According to one particular embodiment of the invention, the oily phase contains at least one silicone gum (CTFA name: dimethiconol), such as the product sold under the name Dow Corning 1503 by the company Dow Corning. When it is present, the silicone gum may be in an amount ranging from 0.01% to 5% by weight and preferably from 0.1% to 3% by weight relative to the total weight of the composition.

The oily phase is present in the composition according to the invention in an amount generally ranging from 10% to 60% and preferably from 15% to 50% by weight relative to the total weight of the composition, this amount comprising the oil(s) and all the lipophilic compounds such as the silicone surfactant and the grafted silicone polymer.

Aqueous Phase

The aqueous phase of the composition of the invention may for example range from 40% to 90% by weight and preferably from 50% to 85% by weight relative to the total weight of the composition. It contains water and, besides the primary alcohol and the polyols, the water-soluble or water-dispersible adjuvants that may be present.

Adjuvants

In a known manner, the composition of the invention may also contain other adjuvants that are common in cosmetics and/or dermatology, for instance active agents, preserving agents, antioxidants, complexing agents, pH regulators (acidic or basic), fragrances, fillers, bactericides, odour absorbers, dyestuffs (pigments and dyes), electrolytes such as magnesium sulfate, and also lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles. Needless to say, a person skilled in the art will take care to select the optional adjuvant(s) to be added to the composition according to the invention and the amounts thereof, such that the advantageous properties intrinsically associated with the composition in accordance with the invention are not, or are not substantially, adversely affected by the envisaged addition.

As fillers that may be used in the composition of the invention, examples that may be mentioned include the pigments such as titanium oxide, zinc oxide or iron oxide and organic pigments; kaolin; silica; talc; boron nitride; organic spherical powders, fibres; and mixtures thereof. Examples of organic spherical powders that may be mentioned include polyamide powders and especially Nylon® powders such as Nylon-1 or Polyamide 12, sold under the name Orgasol by the company Atochem; polyethylene powders; Teflon®; microspheres based on acrylic copolymers, such as those made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; powders of natural organic materials such as starch powders, especially of maize starch, wheat starch or rice starch, which may or may not be crosslinked, such as the starch powders crosslinked with octenyl succinate anhydride, sold under the name Dry-Flo by the company National Starch. Examples of fibres that may be mentioned are polyamide fibres such as especially Nylon 6 fibre (or Polyamide 6) (CTFA name: Nylon 6), Nylon 6.6 fibre (or Polyamide 66) (CTFA name: Nylon 66) or such as poly-p-phenylene terephthamide fibres; and mixtures thereof. These fillers may be present in amounts ranging from 0.01% to 15% by weight, preferably from 0.05% to 10% by weight and even better from 0.1% to 5% by weight relative to the total weight of the composition.

Active agents that may be used especially include vitamins (A, B3, C, E, K, PP, etc.), alone or as a mixture, and also derivatives thereof; keratolytic and/or desquamating agents such as salicylic acid and its derivatives, α-hydroxy acids, for instance lactic acid, citric acid, glycolic acid, and ascorbic acid and its derivatives; antiinflammatory agents; calmatives such as allantoin; depigmenting agents; xanthic bases such as caffeine; tensioning agents such as synthetic polymers, plant proteins, polysaccharides of plant origin optionally in the form of microgels, starches, wax dispersions, mixed silicates and colloidal particles of mineral fillers; mattifying agents; anti-wrinkle agents; refreshing agents; and mixtures thereof.

According to one particular embodiment of the invention, the composition contains at least one refreshing agent that reinforces the refreshing effect provided by the presence of alcohol. This agent may be chosen, for example, from menthol and its derivatives, and menthoxypropanol, and mixtures thereof. When they are present, these agents may be in an amount ranging from 0.001% to 5% by weight and preferably from 0.01% to 2% by weight relative to the total weight of the composition.

The sunscreens may be chosen from organic UV-screening agents, such as the following compounds:
Salicylic derivatives, and especially ethylhexyl salicylate (or ethylhexyl salicylate) sold under the trade name Neo Heliopan OS by Haarmann and Reimer;
Dibenzoylmethane derivatives, and especially butyl methoxydibenzoylmethane sold in particular under the trade name Parsol 1789 by Hoffmann LaRoche;
Cinnamic derivatives, and especially ethylhexyl methoxycinnamate sold in particular under the trade name Parsol MCX by Hoffmann LaRoche;
β,β′-Diphenylacrylate derivatives, and especially octocrylene (2-ethylhexyl α-cyano-β,β-diphenylacrylate), sold in particular under the trade name Uvinul N539 by BASF;
Phenylbenzimidazolesulfonic acid;
Benzylidenecamphor derivatives, and especially terephthalylidenedicamphorsulfonic acid manufactured under the name Mexoryl SX by Chimex, and 4-methylbenzylidenecamphor sold under the trade name Eusolex 6300 by Merck;
Benzophenone derivatives, and especially Benzophenone-3 or Oxybenzone sold under the trade name Uvinul M40 by BASF; benzophenone-4 sold under the trade name Uvinul MS40 by BASF; benzophenone-5;
Phenylbenzimidazole derivatives, and especially Benzimidazilate sold under the trade name Neo Heliopan AP by Haarmann and Reimer;
Triazine derivatives, and especially anisotriazine sold under the trade name Tinosorb S by Ciba Geigy; ethylhexyltriazone sold in particular under the trade name Uvinul T150 by BASF; and diethylhexylbutamidotriazone sold under the trade name Uvasorb HEB by Sigma 3V;
Methylenebis(benzotriazolyl)tetramethylbutyl phenol;
Phenylbenzotriazole derivatives, and especially drometrizole trisiloxane sold under the trade name Silatrizole by Rhodia Chimie; or methylenebis(benzotriazolyl)tetramethylbutylphenol, sold in solid form under the trade name Mixxim BB/100 by Fairmount Chemical, or in micronized form as an aqueous dispersion under the trade name Tinosorb M by Ciba Specialty Chemicals;
and mixtures thereof.

The sunscreens may also be chosen from physical sunblocks. As examples of physical sunblocks that may be added to the composition of the invention, mention may be made of pigments and nanopigments of coated or uncoated metal oxides, especially titanium oxide, iron oxide, zirconium oxide, zinc oxide or cerium oxide, and mixtures thereof, these oxides possibly being in the form of optionally coated microparticles or nanoparticles (nanopigments).

The amount of active agents depends on the desired aim. The active agent(s) may be present, for example, in a concentration ranging from 0.001% to 20%, preferably from 0.01% to 10% and better still from 0.05% to 5% by weight relative to the total weight of the composition.

The composition according to the invention may be in any form whatever, and especially in the form of a more or less thick cream or a fluid of milk type. It finds its application in any topical application, especially in cosmetics and dermatology and more particularly as a cosmetic composition, in particular for treating the skin, in particular for caring for, protecting and/or making up the body skin. It is particularly suitable for giving body skin a coating effect, as explained previously, especially on the arms, the legs, the hands, the feet and the trunk.

Thus, a subject of the present invention is the cosmetic use of a cosmetic composition as defined above, for caring for, protecting and/or making up the skin, especially body skin.

A subject of the present invention is also a process for giving the skin, especially body skin, a coating effect, which consists in applying to the skin a cosmetic composition as defined above.

The examples below of compositions according to the invention are given as illustrations and with no limiting nature. The amounts therein are given as percentages by weight, unless otherwise mentioned.

### EXAMPLE 1

### Body Cream

A. Oily phase

| | |
|---|---|
| cyclopentasiloxane | 12% |
| cyclohexasiloxane | 3% |
| polysilicone-8 | 0.3% |
| cyclopentasiloxane/PEG/PPG-18/18 | 13% |
| dimethicone/water (88/10/2) (Q2-5225C) | |
| (i.e. 1.3% of silicone emulsifier) | |

B. Aqueous phase

| | |
|---|---|
| glycerol | 7% |
| magnesium sulfate | 0.7% |
| denatured ethanol | 30% |

| | | |
|---|---|---|
| water | qs | 100% |

Procedure: Each phase is prepared and the aqueous phase is then stirred slowly and with vigorous stirring into the oily phase.

The composition obtained is a cream with a viscosity of 16 poises (1.6 Pa.s) at 25° C., measured using a Rheomat 180 viscometer at a shear rate of 200 s⁻¹, with a No. 3 spindle.

It is in the form of a translucent, soft, shiny cream, which feels fresh when applied and gives a very good coating effect after application to the body, for example to the arms and legs.

### EXAMPLE 2

### Fluid Composition for the Body

A. Oily phase

| | |
|---|---|
| cyclopentasiloxane | 15% |
| cyclohexasiloxane | 10% |
| polysilicone-8 | 0.5% |
| cyclopentasiloxane/PEG/PPG-18/18 | 13% |
| dimethicone/water (88/10/2) (Q2-5225C) | |
| (i.e. 1.3% of silicone emulsifier) | |

B. Aqueous phase

| | |
|---|---|
| glycerol | 15% |
| magnesium sulfate | 0.7% |
| denatured ethanol | 18% |
| menthol | 0.05% |
| water qs | 100% |

Procedure: Identical to that of Example 1.

The composition obtained is fluid and has a viscosity of 218 cPoises (218 mPa.s), measured at 25° C. using a Rheomat 180 viscometer at a shear rate of 200 s⁻¹, with a No. 2 spindle.

This fluid is translucent, soft and shiny; in addition, it feels fresh when applied to the skin and gives a very good coating effect after application to the body, for example to the arms and legs.

### EXAMPLE 3

### Body Cream

A. Oily phase

| | |
|---|---|
| cyclopentasiloxane | 12% |
| cyclohexasiloxane | 3% |
| polysilicone-8 | 0.5% |
| cyclopentasiloxane/PEG/PPG-18/18 | 13% |
| dimethicone/water (88/10/2) (Q2-5225C) | |
| (i.e. 1.3% of silicone emulsifier) | |
| dimethicone/dimethiconol | 2% |

B. Aqueous phase

| | |
|---|---|
| glycerol | 7% |
| magnesium sulfate | 0.7% |
| denatured ethanol | 20% |
| water qs | 100% |

Procedure: Identical to that of Example 1.

A thick, translucent, soft, shiny cream is obtained, which feels fresh on application and gives a very good coating effect after application to the body, for example to the arms and legs.

### EXAMPLE 4

### Fluid Composition for the Body

A. Oily phase

| | |
|---|---|
| cyclopentasiloxane | 15% |
| cyclohexasiloxane | 10% |
| polysilicone-8 | 0.5% |
| cyclopentasiloxane/PEG/PPG-18/18 | 13% |
| dimethicone/water (88/10/2) (Q2-5225C) | |
| (i.e. 1.3% of silicone emulsifier) | |

B. Aqueous phase

| | |
|---|---|
| glycerol | 15% |
| magnesium sulfate | 0.7% |
| denatured ethanol | 18% |
| silica | 2% |
| water qs | 100% |

Procedure: Identical to that of Example 1.

The composition obtained is fluid and has a viscosity of 210 cPoises (210 mPa.s), measured at 25° C. using a Rheomat 180 viscometer at a shear rate of 200 s⁻¹, using a No. 2 spindle.

This fluid is translucent, soft and shiny; in addition, it feels fresh when applied to the skin and gives a very good coating effect after application to the body, for example to the arms and legs.

The above written description of the invention provides a manner and process of making and using it such that any person skilled in this art is enabled to make and use the same, this enablement being provided in particular for the subject matter of the appended claims, which make up a part of the original description and including a composition comprising:
an aqueous phase,
an oily phase comprising at least one silicone oil,
at least one silicone emulsifier,
at least one grafted silicone polymer comprising a polysiloxane portion and a non-silicone organic chain portion, one of the two portions constituting the main chain of the polymer and the other being grafted onto the main chain,
at least 15% by weight of at least one C₂-C₃ primary alcohol relative to the total weight of the composition, and
at least 5% by weight of at least one polyol relative to the total weight of the composition,
   wherein said composition is in the form of a water-in-oil emulsion.

As used above, the phrases “selected from the group consisting of,” “chosen from,” and the like include mixtures of the specified materials.

As used herein, where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising:
an aqueous phase,
an oily phase comprising at least one silicone oil,
at least one silicone emulsifier,
at least one grafted silicone polymer comprising a polysiloxane portion and a non-silicone organic chain portion, one of the two portions constituting the main chain of the polymer and the other being grafted onto the main chain,
at least 15% by weight of at least one C₂-C₃ primary alcohol relative to the total weight of the composition, and
at least 5% by weight of at least one polyol relative to the total weight of the composition,
wherein said composition is in the form of a water-in-oil emulsion.

2. The composition according to claim 1, wherein the total amount of polyols and silicone oils ranges from 25% to 60% by weight relative to the total weight of the composition.

3. The composition according to claim 1, comprising ethanol.

4. The composition according to claim 1, wherein the primary alcohol is present in an amount ranging from 15% to 40% by weight relative to the total weight of the composition.

5. The composition according to claim 1, wherein the amount of silicone emulsifier ranges from 0.1% to 5% by weight relative to the total weight of the composition.

6. The composition according to claim 1, comprising a grafted silicone polymer that is a polydimethylsiloxane onto which is grafted, via a thiopropylene linker, mixed polymer units of poly(meth)acrylic acid and/or poly(alkyl (meth)acrylate.

7. The composition according to claim 1, wherein the grafted silicone polymer is present in an amount ranging from 0.1% to 5% by weight relative to the total weight of the composition.

8. The composition according to claim 1, wherein the amount of polyol ranges from 5% to 30% by weight relative to the total weight of the composition.

9. The composition according to claim 1, comprising at least one volatile silicone oil.

10. The composition according to claim 1, wherein the amount of silicone oil ranges from 5% to 55% by weight relative to the total weight of the composition.

11. The composition according to claim 1, wherein the amount of oily phase ranges from 10% to 60% by weight relative to the total weight of the composition.

12. The composition according to claim 1, further comprising at least one silicone gum.

13. The composition according to claim 1, further comprising at least one refreshing agent selected from the group consisting of menthol, menthol derivatives, and menthoxypropanediol.

14. The composition according to claim 1, wherein said composition is a cosmetic composition.

15. The composition according to claim 1, wherein said composition is translucent.

16. The composition according to claim 1, comprising a grafted silicone polymer obtained by radical copolymerization between, on the one hand, at least one non-silicone anionic organic monomer having ethylenic unsaturation and/or a non-silicone hydrophobic organic monomer having ethylenic unsaturation, and, on the other hand, a silicone having in its chain at least one functional group capable of reacting with the ethylenic unsaturations of the non-silicone monomers.

17. The composition according to claim 3, wherein the total amount of polyol(s) and of silicone oil(s) ranges from 25% to 60% by weight relative to the total weight of the composition.

18. The composition according to claim 1, further comprising at least one additive selected from the group consisting of sunscreens, active agents, preserving agents, antioxidants, complexing agents, pH regulators, fragrances, fillers, bactericides, odour absorbers, dyestuffs, and electrolytes.

19. A method for caring for, protecting and/or making up the skin, comprising applying the composition of claim 1 to the skin.

20. A method for giving the skin a coating effect, comprising applying to the skin the composition according to claim 1.
